(19) United States
(12) Patent Application Publication
(10) Pub. No.: US 2022/0390422 A1
(43) Pub. Date: Dec. 8, 2022

(54) DETECTION OF TESTING ERRORS IN LATERAL FLOW ASSAYS

(71) Applicant: Clas Sivertsen, Lilburn, GA (US)

(72) Inventor: Clas Sivertsen, Lilburn, GA (US)

(21) Appl. No.: 17/339,037

(22) Filed: Jun. 4, 2021

(51) Int. Cl. G01N 31/22; G01N 33/52

(52) U.S. Cl. CPC G01N 31/22; G01N 33/523; G01N 2333/928

(57) ABSTRACT

A lateral flow assay (“LFA”) strip for receiving a test sample from a domain. The LFA strip includes a test line on the LFA strip that indicates a positive result in the presence of a chemical-of-interest being tested for in the domain and a negative result in the absence of the chemical-of-interest being tested for in the domain. The strip also includes a verification line on the LFA strip that indicates a positive result when a target marker present in the domain is on the LFA strip.

### BACKGROUND

The present invention generally relates to testing and more specifically, to a test strip for detecting errors when using lateral flow assays.

Coronavirus Disease 2019 (“COVID-19”) is spreading throughout the world caused by the spread of a novel coronavirus called SARS-CoV-2. With the rapid spread of the disease, testing quickly, accurately, and efficiently is becoming more important. Testing is often performed by collecting a sample via swabbing the back of the nasal cavity with a nasopharyngeal swab, swabbing the nasal cavity with a nasal swab, or swabbing the throat of a patient and performing an analysis of the collected sample.

As the importance of tests increases, for example, gaining entry to a workplace or an airplane may require testing negative to COVID-19, for example, there are growing incentives for people exhibiting symptoms to perform a fraud on the testing equipment. In addition, testing of other domains, such as environmental and food conditions, also provides opportunities for fraudulent testing. Testing for other pathogens is also important, as well as testing of a person's or an animal's immune response to natural infection or vaccination as mutations give rise to new variants of viruses, bacteria, and parasites.

### SUMMARY

Embodiments of the present invention are directed to a lateral flow assay (“LFA”) strip for receiving a test sample from a domain. The LFA strip includes a test line on the LFA strip that indicates a positive result in the presence of a chemical-of-interest being tested for in the domain and a negative result in the absence of the chemical-of-interest being tested for in the domain. The strip also includes a verification line on the LFA strip that indicates a positive result when a target marker present in the domain is on the LFA strip. Although LFA strips can be used standalone, they are often placed in a plastic cassette, a tube, or a cardboard housing to protect the strip from direct contact with the environment, fingers, or other chemicals or potentially interfering pollutants in the environment.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 illustrates an LFA strip having one or more of a verification line, control line, test line, a conjugate pad, and an analyte pad consistent with the principles of the present invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two- or three-digit reference numbers. With minor exceptions, the leftmost digit(s) of each reference number corresponds to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Turning now to an overview of technologies that are more specifically relevant to aspects of the invention, as previously stated, SARS-CoV-2 is spreading rapidly around the country and around the world resulting in a large portion of the population being at risk of developing COVID-19. It is imperative to test, often repeatedly, the population for SARS-CoV-2, and present testing systems fail to adequately check for fraud.

Present testing systems often require a nasal swab to get samples from a patient. Some tests exist that involve saliva, but those tests, like the nasal tests, also rely on actual material from the patient. These tests may be performed by the patient themselves, without the presence of a nurse, doctor, lab technician, or other witness. Such tests are rife with opportunities for fraud, as a patient may surreptitiously not use swabbed mater from the inside of a nose or actual saliva from inside her mouth. The pressing matter of boarding a plane or going to work may make even good people susceptible to the easy fraud of using water instead of material gathered from the patient.

During testing, samples are often taken in a rush or incorrectly, so that fraud is not the only issue. Mistakes can happen due to human error or other factors where a sample does not sufficiently collect enough material to produce a reliable result.

Additionally, testing systems may be used in multiple domains. The domain, mentioned above, is the human domain, but testing is also performed in other domains, such as animals and the environment, for example. Reference to a domain herein is reference to any situation, for example, human testing, animal testing, environmental testing, and food testing, that provides an opportunity for fraudulent testing by not testing the actual substance that is allegedly being tested.

Turning now to an overview of the aspects of the invention, one or more embodiments of the invention address the above-described shortcomings of the prior art by providing a lateral flow assay (“LFA”) strip. The LFA strip provides indicators (stripes at various places along the LFA strip) that indicate the presence of analytes present in a sample. In addition to lines indicating presence of tested analytes in the sample, an additional line is placed on the LFA strip. This “verification line,” as it is termed herein, reacts to target markers, for example, a protein produced by a gland in the body, an antigen, a neutralizing or non-neutralizing antibody, a pathogen or a component of a pathogen, such as a protein, or a non-biological chemical marker such as a drug, substance, or vitamin. For example, in a urine based test the verification line would react and provide a visual indication of urea. Further examples include reacting to amylase, lipase or lingual lipase in a saliva based test, and mucin proteins in a nasal swab test. In a human testing domain, for example, water on the LFA strip would not produce any result on the verification line, but the proper protein for the type of test administered would produce a visible line on the verification line that would demonstrate that actual material produced by the body was used on the LFA strip.

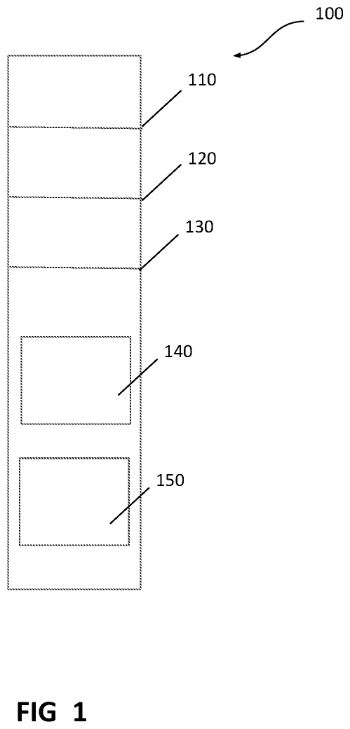

FIG. 1 illustrates an LFA strip 100 having at least one verification line 110, control line 120, test line 130, conjugate pad 140, and analyte pad 150 consistent with embodiments of the present invention. An analyte containing a sample from the domain is placed on the analyte pad 150. The analyte flows up the LFA strip 100 through the area of the strip having the conjugate pad 140, test line 130, control line 120, and verification line 120.

The material of the test line 130 provides a positive result in the presence of a chemical-of-interest being tested for in the domain and a negative result in the absence of the chemical-of-interest being tested for in the domain. Where the chemical-of-interest can be a pathogen, or a piece of a pathogen, a biological marker, such as a protein of a chemical organic or inorganic, and where the biological marker can specifically be a substance such as the active ingredient in a drug, food additive, or environmental pollutant.

The material of the verification line 110 responds to a target marker that should be found within the domain. For example, in a test taken from the nasal cavity, the material of the verification line 110 will react to amylase found in the nasal cavity. If amylase is present, the verification line 110 will turn a color so that the person reviewing the test can tell that the material placed on the analyte pad 150 actually came from a nasal cavity.

In another example, if the material on the analyte pad should be urine, the material of the verification line 110 would respond to the presence of uric acid or urea before turning color. As yet another example, for saliva tests, the material of the verification line 110 may respond to amylase that breaks down starches into sugars or to lingual lipase which breaks down fat.

In another embodiment consistent with the principles of the present invention, the verification line 110 also acts as a control line, eliminating the need for control line 120. In this embodiment verification line 110 “becomes” a first color, for example red, in the presence of the sample when a target marker within the domain is not present, and another color for example green when the target marker within the domain is present.

While the description to this point has been with respect to human or animal testing, LFA strips may also be used in testing outside of human or animal testing in other domains. For example, pool water, ocean water, industrial outflows, food samples, wastewater, and chemical solutions may need to be tested for contaminants or other substances. In order to ensure that the allegedly tested substance was tested, and not merely distilled water for example, an LFA strip consistent with an embodiment of the present invention may have a verification line that does not respond to a target biological marker, but rather responds to a marker that indicates authenticity of the allegedly tested substance. For example, when testing sea water, the verification line responds to salinity consistent with salinity of the oceans.

After reading this detailed description, those skilled in the art will appreciate that other materials could be used in the verification line 110 that would react to proteins whose presence would indicate a proper test using animal proteins, and not just water placed on the test strip.

The verification line 110 on the LFA strip may also be designed to change conductivity in the presence of the target marker, and the conductivity has a relationship, for example, proportionality, to the concentration of the target marker. Furthermore, the test line 130 may change conductivity in the presence of the chemical-of-interest, and the conductivity has a relationship, for example, proportionality, to the concentration of the chemical-of-interest.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A lateral flow assay (“LFA”) strip for receiving a test sample from a domain, comprising:
a test line on the LFA strip that indicates a positive result in the presence of a chemical-of-interest being tested for in the domain and a negative result in the absence of the chemical-of-interest being tested for in the domain; and
a verification line on the LFA strip that indicates a positive result when a target marker present in the domain is on the LFA strip.

2. The LFA strip of claim 1, wherein the target marker is amylase.

3. The LFA strip of claim 1, wherein the target marker is uric acid.

4. The LFA strip of claim 1, wherein the target marker is selected from the group consisting of lingual lipase and mucin.

5. The LFA strip of claim 1 further comprising an analyte pad placed on the LFA strip.

6. The LFA strip of claim 5, wherein the analyte pad is in liquid communication with the verification line and the test line.

7. The LFA strip of claim 6, wherein the test line is placed between the verification line and the analyte pad.

8. The LFA strip of claim 1, wherein the target marker is a substance found in a nasal cavity.

9. The LFA strip of claim 1, wherein the target marker is found in urine.

10. The LFA strip of claim 1, wherein the target marker is found in a mouth.

11. The LFA strip of claim 8, wherein an analyte pad is in liquid communication with the verification line and the test line.

12. The LFA strip of claim 9, wherein an analyte pad is in liquid communication with the verification line and the test line.

13. The LFA strip of claim 10, wherein an analyte pad is in liquid communication with the verification line and the test line.

14. The LFA strip of claim 11, wherein the test line is placed between the verification line and the analyte pad.

15. The LFA strip of claim 12, wherein the test line is placed between the verification line and the analyte pad.

16. The LFA strip of claim 13, wherein the test line is placed between the verification line and the analyte pad.

17. The LFA strip of claim 1, wherein the verification line on the LFA strip that indicates a positive result when a target marker present in the domain on the LFA strip produces a first result color when an analyte is present at the verification line that contains the target marker and a second result color when an analyte is present at the verification line that does not contain the target marker.

18. The LFA strip of claim 1, wherein the verification line on the LFA strip changes conductivity in the presence of the target marker.

19. The LFA strip of claim 18, wherein the conductivity is proportional to the concentration of the target marker.

20. The LFA strip of claim 1, wherein the test line on the LFA strip changes conductivity in the presence of the chemical-of-interest.
